(19) United States
(12) Patent Application Publication
(10) Pub. No.: US 2005/0255054 A1
(43) Pub. Date: Nov. 17, 2005

(54) Method for tooth whitening, in particular a system for tooth whitening using a dissolvable tray or strip

(76) Inventors: Graham K. Philp JR., Naples, FL (US); Damon Brown, Boston, MA (US)

Correspondence Address: SHLESINGER, ARKWRIGHT & GARVEY LLP, 1420 KING STREET, SUITE 600, ALEXANDRIA, VA, 22314, US

(21) Appl. No.: 11/118,759

(22) Filed: May 2, 2005

Related U.S. Application Data
(60) Provisional application No. 60/566,431, filed on Apr. 30, 2004.

(51) Int. Cl. A61K 7/20; A61C 5/00

(52) U.S. Cl. 424/53; 433/217.1

(57) ABSTRACT

Method of tooth whitening includes the use of a tooth whitening composition incorporated in a dissolvable strip or mouth tray. This strip or mouth tray may be made of different compositions, one example being a hydrogel including acemannan, such as an Acemannan Hydrogel™ from Carrington Industries. The strip or mouth tray may incorporate an oxidizing agent, which is encapsulated within a water-soluble substance, which dissolves over time, thus providing a time-release effect. The rate of time release of the whitener may be varied depending on the intended use.

### CROSS-REFERENCE TO RELATED APPLICATION

This application claims the priority of application No. 60/566,431, filed Apr. 30, 2004, which is incorporated herein by reference.

### FIELD OF THE INVENTION

The present invention relates to tooth whitening. More particularly, the invention relates to a tooth whitening composition, and a system and method for tooth whitening using a dissolvable tray or strip.

### BACKGROUND OF THE INVENTION

Tooth structures, such as enamel, dentin, and the acquired pellicle, become discolored with age, owing to blood, amalgam restoration, antibiotics, substances in food, beverages, and tobacco

Tooth enamel is predominately formed from an inorganic substance, hydroxyapatite crystals along with approximately 5% organic materials, predominately collagen.

The dentin is composed of about 20% protein including collagen, the balance including organic materials and hydroxyapatite crystals.

The acquired pellicle is a proteinaceous layer on the surface of the tooth enamel, which layer can be removed by mechanical expedients such as intensive tooth cleaning.

Tooth bleaching to achieve lighter (i.e., whiter) teeth, is considered to be very desirable in today's cosmetically—orientated society. Tooth bleaching has been generally accomplished by gels, pastes, or liquids, which typically contain an oxidizing agent such as hydrogen peroxide that can form an oxygen anion (free radical or nascent oxygen species), which will attack the chromogen (stain) molecules, making them colorless and/or water soluble or both resulting in the teeth being lighter and brighter appearing.

The most commonly used oxidizing agent is hydrogen peroxide, which is obtained in a liquid form, and which is mixed with an anhydrous carrier containing glycerin and/or propylene glycol and/or polyethylene glycol.

Also commonly used is carbamide peroxide (urea hydrogen peroxide) which can be dissolved in ethanol and which upon contact with water dissociates into urea and hydrogen peroxide. The hydrogen peroxide in the presence of water then dissociates into water and nascent oxygen (free radical). This nascent oxygen is highly reactive and reacts with the stain molecules making them water soluble or transparent, or both.

The greatest oxidizing activity is required at the tooth surface and within the first few millimeters of the enamel or dentin. There have been known delivery systems to deliver the oxidizing agent to the surfaces of the teeth.

A common approach is to have a dental professional construct a custom-made tray from an impression to provide in a cast of the patient's teeth. The oxidizing gel is dispensed into the custom-made tray by the patient and the tray is worn over the teeth intermittently for a period of two weeks to several months depending on the severity of the staining. This approach can cause sensitivity in over 50% of the patients and it can be uncomfortable for the patients to wear the tooth bleaching trays. Tooth sensitivity is believed to result from movement of fluid through the dentinal tubules, which is sensed by nerve endings within the tooth. The carriers propylene glycol and polyethylene glycerin contribute to the tooth sensitivity often experienced by wearing a bleaching tray.

Another approach is to incorporate the oxidizing agent into a strip and have the patient wear the strip intermittently over a period of two weeks. This approach also has the disadvantage of being awkward for the patients to wear, and many patients wearing the strips also experience tooth sensitivity. Further, the strips have an inherent problem of contacting the teeth only on the facial surfaces and most staining occurs in between the teeth in the interproximal areas, where it can be difficult to place the strip. Thus, the stained interproximal areas of the tooth remain unwhitened.

Another recent approach is simply to paint the oxidizing agent, carbamide peroxide or hydrogen peroxide, directly on the tooth. In products containing glycerin and/or propylene glycol or polyethylene glycol, tooth sensitivity can be experienced. Further, these products are gels, and the gels can easily be removed by the lips and tongue decreasing their effectiveness.

One approach dissolves the carbamide peroxide in alcohol with a resin, and when the alcohol is allowed to evaporate away, the resin forms a film or precipate, which is left on the surface of the teeth. When saliva contacts the teeth, the peroxide is release in a relatively strong concentration for a period of up to 20 minutes. This product contains no glycerin or propylene glycol or polyethylene glycol, and tooth sensitivity is virtually non-existent.

These products have yielded results; however, they take approximately two weeks of use several times a day to see the desired result.

To overcome the problem of taking excessive time to whiten teeth, manufacturers developed an approach which is used by a dental professional in the dental office, and which can result in whitening the teeth in approximately one hour. These systems generally use oxidizing compositions (hydrogen peroxide up to 35% to 40%) which are applied directly on the teeth in a dental office under the supervision of a dentist or hygienist. Owing to the high concentration of oxidizing agents contained in these office products, they can be hazardous to the patient if not handled carefully, the patients soft tissues—gingiva, lips and tissues—must be isolated from potential exposure to the strong concentration of the oxidizing agent by the use of a rubber dam or by covering the soft tissues with a polymerizable resin that is shaped to conform to the gingival contours. The thus isolated oxidizing composition is subsequently cured by a high intensity light source directed at the teeth on which the oxidizing composition has been provided.

Known patents directed to these various tooth whitening systems include:
U.S. Pat. No. 4,952,143 to Becker et al.;
U.S. Pat. No. 5,032,178 to Cornell; and
U.S. Pat. No. 5,785,527 to Jensen et al.

There is a need for a rapid efficient tooth whitening system, method, and composition which is capable of whitening the teeth with a low concentration of oxidizing agent, so that the patient can use the product at home without harming the tissues or burning the tissues or causing mouth/tooth irritation or tooth sensitivity.

There are several drawbacks of conventional paint-on whitening. First the bottles must be thoroughly shaken, for there is a tendency for the whitening compositions to settle out. Further the paint on products are messy and many people drool while painting the products on their teeth getting the oxidizing agents on their clothing and counters, resulting in their clothes and fixtures being discolored. Also the bottles are somewhat porous and the volatile alcohol can escape, resulting in lighter concentrations of product than is desirable. This can occur more rapidly when the cap is inadvertently not replaced tightly.

Traditional strips also have many disadvantages in that they are very awkward to wear, often causing excessive drooling and difficulty with speech. Traditional strips are also difficult for many people to place on in a manner which ensures the strip is in contact with the interproximal areas where most of the stain occurs.

There is a need for a rapid and efficient tooth whitening system, method, and composition which is very convenient to apply and can utilize a strip which wraps around the facial and lingual surfaces of the teeth and be made of a material which will dissolve and be healthy for the teeth and gums. It may be ideal to have the dissolvable strip be completely biodegradable over a 6-8 hour period.

| Drawbacks of Paint on | Drawbacks of Traditional |
|---|---|
| Whitening | Strips |
| Messy | Not getting whitening agent |
| | in between interproximals |
| Drooling | Awkward to wear |
| Getting on Clothing | Not achieving whitening of |
| | interproximals |

### OBJECTS AND SUMMARY OF THE INVENTION

It is an object of the invention to provide a tooth whitening method and system that overcomes the drawbacks of prior art methods, systems, and devices.

It is another object of this invention to provide a tooth whitening system which the consumer can use at home or anywhere, thereby being easier to use and more cost-effective than conventional strips.

It is further object of the invention to utilize a dissolvable mouth tray or strip, which converts from a freeze-dried solid to a sticky gel upon contact with water or moisture.

It is a further object of the invention to have the gel film of the mouth tray or strip be completely biodegradable over a 6-8 hour period.

It is a further object of the invention to have the mouth trays or strips coated on one side with a biodegradable material, which will not stick to the lips or cheeks for an initial period of time, such as about the first ½ hour after application of the tray or strip to the teeth.

It is a further object of the invention to have the mouth tray or strip be made of a material, which is healthy for the gums and tissues.

It is a further object of the invention to have the oxidizing agent encapsulated in a dissolvable substance so that the oxidizing agent can be incorporated into the freeze-dried gel.

It is a further object of the invention to have the oxidizing agent encapsulated in a biodegradable substance so that the oxidizing agent can be incorporated into the freeze-dried gel.

It is a further object of the invention to have the oxidizing agent be encapsulated to give a time-release effect.

It is another object of the invention to provide a liquid oxygenating agent, such as hydrogen peroxide, and to incorporate the liquid agent in a strip or mouth tray prior to formulating the freeze-dried state (i.e., prior to the steps of freeze drying).

It is a further object of the invention to incorporate a solid form of a phase transfer catalyst incorporated into the freeze-dried gel. This may be in the form of a resin, which would enhance the stickiness of the gel.

Another object of the invention is to provide a compound in the mouth tray or strip that has properties beneficial to mouth tissue, and which soothes, heals, and oxygenates mouth tissue (e.g., the gums).

Another object of the invention is to provide Acemannan Hydrogel™ in the mouth tray or strip that has properties beneficial to mouth tissue, and which soothes, heals, and oxygenates mouth tissue (e.g., the gums).

The invention includes a tooth-whitening device which incorporates a time-release feature so that a whitening compound carried by the strip or mouth tray whitens a user's teeth over a period of time.

The inventive device includes one of a mouth tray or strip containing a time-release compound in which a tooth whitener is contained, such as by encapsulation, for releasing the whitening agent over time.

The invention likewise includes a dissolvable strip which contains an oxidizing agent, along with a phase transfer catalyst for enhancing the whitening of the teeth effected by the oxidizing agent.

The invention further includes a tooth whitening strip which is dissolvable over a predetermined period of time, so that a user may wear the whitening strip, and the whitening strip governs the rate and the time for which the whitening process is carried out, so that the user is not required to time the whitening process.

The invention includes the use of a freeze-dried hydrogel which incorporates an oxidizing agent, such as carbamide peroxide or sodium perborate monohydrate, which is encapsulated in a water soluble substance, which can result in a time-release effect of the oxidizing agents.

The freeze-dried hydrogel incorporates the oxidizing agent, and is made into a mouth tray or strip, which is simply pressed on the teeth and gums and converts to a sticky gel, which temporarily adheres to the teeth, upon contact with water. The water may be a layer of water/saliva which would typically be on the user's teeth when the user opens his or her mouth to apply the dissolvable whitening strip or dissolvable tray made of such freeze-dried gel incorporating a whitener.

The tray or strip may be coated with an outer layer of material which will not initially adhere to the lips or cheeks. For example, the outer coating may be selected to prevent sticking of the tray or strip to the lips or cheeks for a sufficiently long period of time so that a predetermined amount (e.g., the majority) of the encapsulated whitener may be freed and react with the outer surface of the tooth before the outer coating wears away or allows adherence or degradation/dissolving of the coated layer owing to contact with moisture from the lips or cheeks.

Relative terms such as up, down, inner, and outer are for convenience only and are not intended to be limiting.

### BRIEF DESCRIPION OF THE DRAWINGS

FIG. 1 illustrates an embodiment of the invention including a strip, shown adhered to an outer surface of a user's tooth;

FIG. 2 illustrates the embodiment of FIG. 1, shown at a later period in time;

FIG. 3 illustrates the embodiment of the strip of FIG. 1, shown at a still later period of time than FIG. 2;

FIG. 4 illustrates the embodiment of FIG. 1, shown at a yet still further point in time later than FIG. 3;

FIG. 5 illustrates another embodiment of the invention in which a tray, such as a dissolvable or biodegradable tray, is in use in a manner similar to the embodiment of FIG. 1-4; and

FIG. 6 illustrates an embodiment of another mouth tray according to the invention, which mouth tray may be in the form of a strip.

### DETAILED DESCRIPTION OF THE EMBODIMENTS

These embodiments are set forth to describe and illustrate the invention but are not intended to be limiting.

The invention achieves tooth whitening by using a freeze-dried strip, for example a strip made from a freeze-dried hydrogel available from Carrington Industries. This product contains a high concentration of complex carbohydrates, which are nontoxic, hypoallergenic, and nonirritating. Specifically this product contains acemannan, a complex mannose carbohydrate derived from the Aloe Vera plant that has an inherent stickiness when in contact with moisture and water.

The Carrington product, a freeze-dried gel, is available as Acemannan Hydrogel™, and is responsible for Aloe Vera healing and soothing properties and is the active product derived from Aloe Vera. Further, Acemannan Hydrogel™is a mixture of natural anti-inflammatory and non-inflammatory components. This product is non-stinging, easy to apply and works on contact with water to produce a sticky film, which can be used to provide rapid pain relief of mouth ulcers, canker sores, minor mouth lesions, ulcers and abrasions. This freeze-dried material can be made into a strip or mouth tray which when positioned over the teeth and gums transforms into a gelatinous film which covers and adheres to the teeth and gums. Acemannan Hydrogel™ is an acceptable substrate to form a whitening tray or strip according to the invention that can dissolve, and further this material completely biodegrades in 6-8 hours, and this product may be swallowed by the user. A description of acemannan and Acemannan Hydrogel™ may be found at www.carringtonlabs.com.

To incorporate the oxidizing agent into the Acemannan Hydrogel™ it is contemplated that one will encapsulate the oxidizing agent, such as carbamide peroxide, with a water-soluble substance, for example a cellulose or starch, as is typically done in the pharmaceutical and fertilizer industries. These can be amalgamated in the Acemannan Hydrogel™ and with different thicknesses providing a time-release effect. To enhance the efficiency of the oxidizing agent a solid phase transfer catalyst can also be incorporated in the Acemannan Hydrogel™.

To enhance placement of the Acemannan Hydrogel™ strip the strip may be formed large enough to wrap around the teeth and gums. Thanks to the anti-inflammatory and pro-healing properties of the Acemannan Hydrogel™ such would actually be soothing and healing for the gums. Incorporating an oxidizing agent into the Acemannan Hydrogel™ would be healthy for the gums and may help inflamed gums which have become inflamed from periodontal disease.

The strip or mouth tray applied to the teeth and gums as described may be configured to cause a higher density or amount of the Acemannan Hydrogel™ gelatinous hydrogel to be between the teeth in the interproximal area, in use, and which area is the area where most stains occur. The encapsulated oxidizing agent may be formulated in a relatively low concentration of approximately 3% making it suitable for professional or home use. The efficiency can be enhanced with the use of a phase transfer catalyst, as described in Applicant's co-pending application (Applicant's ref. no. 7354) entitled “Method For Tooth Whitening, In Particular A System For Tooth Whitening Using A Phase Transfer Catalyst (PTC)”, filed Apr. 28, 2005, and which is incorporated herein by reference in its entirety, and the oxidizing agent can be made to release the nascent oxygen species with a time-release mechanism enabling lower concentrations of oxidizing agent to be very effective. The time-release mechanism may be activated as soon as the Acemannan Hydrogel™ transforms to a sticky gel and continues for approximately 30-45 minutes. The hydrogel would completely biodegrade after approximately 6-8 hours.

### Dissolvable Strip or Mouth Tray

An example of a dissolvable mouth tray or mini tray or strip containing an oxidizing agent along with a phase transfer catalyst is as follows.

In this approach a pretreatment rinse would be unnecessary. This inventive system makes use of a dissolvable substance pre-made into mini trays or strips. The dissolvable substance may be, for example, a freeze-dried hydrogel from Carrington Industries containing a high concentration of acemannan, the chemical responsible for aloe vera's healing and soothing properties. The hydrogel may incorporate a phase transfer catalyst along with an existing oxidizing agent (carbamide peroxide) or encapsulated sodium perborate monohydrate granules as an oxidizing agent or encapsulated carbamide peroxide. The encapsulated oxidizing agent may be encapsulated within a water-soluble substance, which would enable the oxidizing agent to give a time-release effect.

The freeze-dried hydrogel may be molded in whatever shape desired such as a mouth tray or a strip form. When provided on the tooth surface(s), and water contacts the freeze-dried hydrogel, it would convert to a sticky gel which would temporarily adhere to the tooth surface. When moisture penetrates through the encapsulated oxidizing agent the phase transfer catalyst would carry the oxygen free radicals to the stain and a very convenient and efficient tooth whitening system would be developed.

The freeze-dried acemannan from Carrington Industries offers several advantages as a carrier substance: it contains acemannan in high concentrations, acemannan being the chemical responsible for the healing and soothing properties of aloe vera. This combination would thus promote healthy gums and tissues along with tooth whitening. Also the Carrington freeze-dried material may readily incorporate a phase transfer catalyst and an encapsulated oxidizing agent and upon contact with moisture the freeze-dried material converts to a sticky gel, which temporarily adheres to the tooth; and further it is completely biodegradable in approximately 6-8 hours. If the oxidizing agent (e.g., sodium perborate monohydrate or carbamide peroxide) were encapsulated with a water soluble substance, this would result in a time release effect of the oxidizing agent, especially in the interproximal areas; i.e., the regions between the teeth.

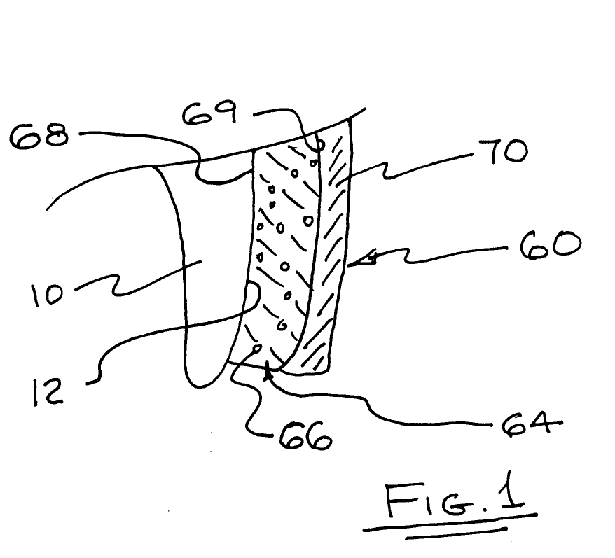

FIGS. 1-4 illustrate an embodiment of the invention in which the teeth are whitened by using a dissolvable or biodegradable strip 60.

Tooth 10 includes an outer surface 12 which will typically be moist owing to the presence of water or saliva when the user opens his or her mouth.

Dissolvable strip 60 includes a layer 64 having tooth whiteners 66 therein. Layer 64 may be a gel layer, such as a freeze-dried gel encapsulating with tooth whiteners 66. Layer 64 includes an inner surface 68 which will adhere to outer surface 12 of tooth 10 thanks to the presence of the water or saliva on the user's teeth.

A further layer 70, which may be a dissolvable or biodegradable layer, may be provided on an outer surface 69 of inner layer 64.

Outer, dissolvable layer 70 may be formulated to dissolve at a slower rate than inner layer 64. In that manner, the tooth whitener 66 will be available to whiten the teeth in the desired manner, as opposed to being released into other parts of the user's mouth. Outer layer 70 may likewise be formulated to dissolve at a faster rate, or at the same rate, as inner layer 64, depending on the intended use.

FIG. 1 illustrates when a user has first adhered strips 60 to outer surface 12 of tooth 10.

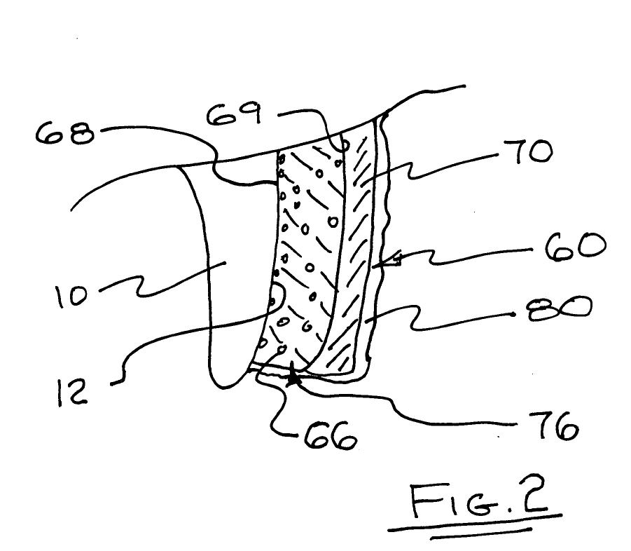

FIG. 2 illustrates a later time than FIG. 1, after strip 60 has been in place on the teeth for a period of time. FIG. 2 shows that the dissolving inner gel layer 76 is releasing whiteners 66 which, in turn, are reacting with and whitening intrinsic and extrinsic stains on outer layer 12 of tooth 10. Saliva 80 is present on layer 70, yet has dissolved or degraded layer 70 relatively little.

Outer layer 12, as will be readily apparent to a person having ordinary skill in the art, may be considered to include the outermost surface of the tooth, as well as the stained outer layers of the tooth to be whitened. Those stained outer layers may be up to the first 2 or 3 millimeters of the depth of the outer layer.

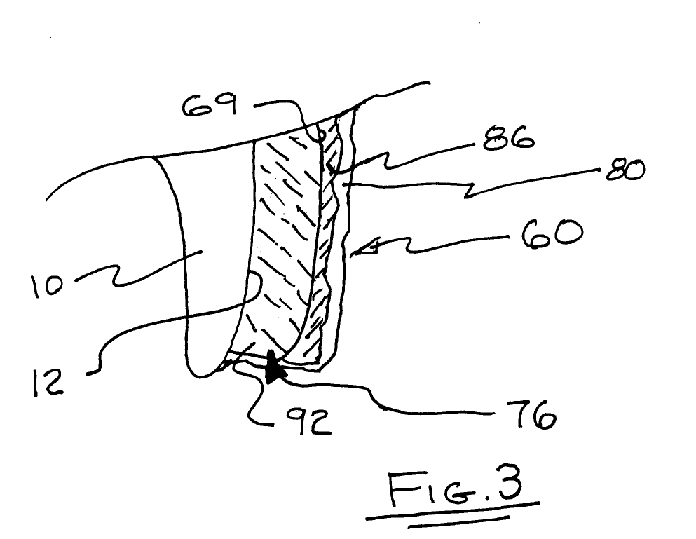

FIG. 3 illustrates a still further point in time, later than shown in FIG. 2, and at which a fully moistened and reacted gel layer 92 is shown adjacent tooth 10 with no remaining whitening agent 66 present to react with the tooth stains on and in layer 12 of tooth 10. The outer layer 70 has begun to dissolve and is shown as a partially dissolved outer layer 86.

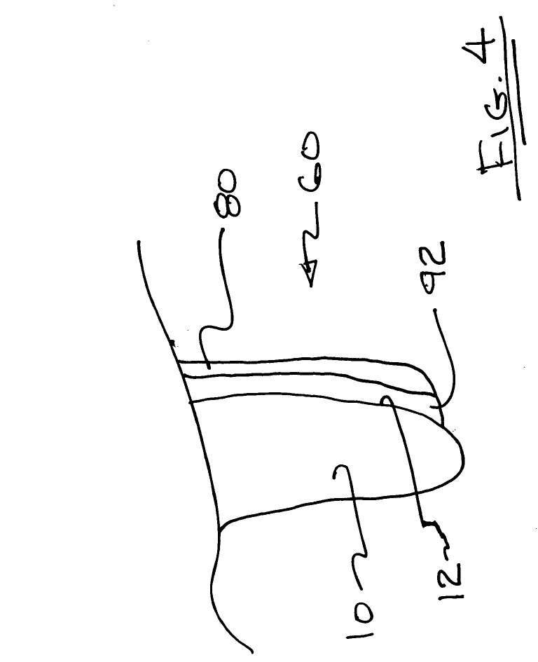

FIG. 4 illustrates a yet still further point in time, later than FIG. 3, in which the strip 60 has almost completely dissolved in the presence of saliva 80 and thanks to the mechanical forces exerted by the lips (and to an extent by the tongue).

After even more time has elapsed than the time period shown in FIG. 4, the strip will be completely gone from the teeth as the strip will have been completely worn away and dissolved. This time may be up to about 6-8 hours, or more, depending on desired characteristics and the intended use.

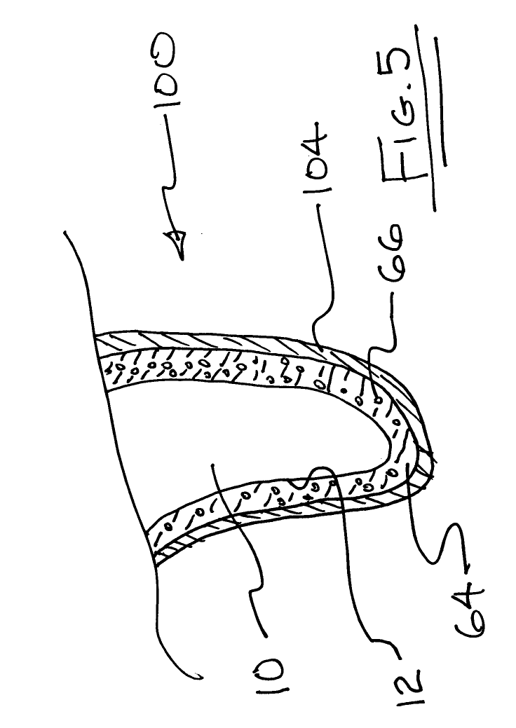

FIG. 5 illustrates an embodiment of the invention in which a dissolvable or biodegradable tray 100 may include a dissolvable or biodegradable outer layer 104 and an inner layer 64 including whitener 66 distributed therein or encapsulated therein, in a manner similar to the embodiment of FIGS. 1-4.

The use of tray 100 will be similar to the use of strip 60, and, when placed on the teeth, the water or saliva present on outer layer 12 of tooth 10 will serve to adhere inner layer 64 to the tooth. Tray 100 may likewise include a dissolvable or biodegradable outer layer 104 which will function in a manner analogous to the functioning of outer layer 70 of strip 10.

The whitening and dissolving process will continue in a manner analogous to that shown and described in connection with the embodiments of FIGS. 1-4 and throughout the text.

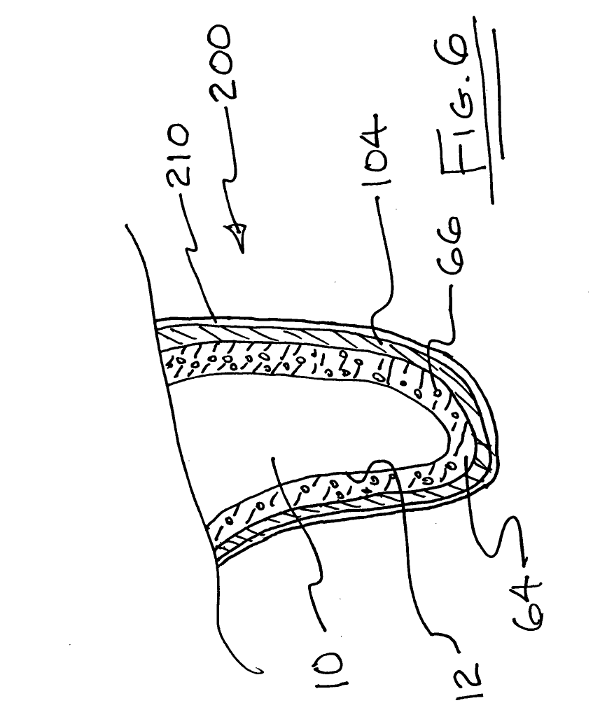

FIG. 6 illustrates an embodiment of a mouth tray 200 according to the invention.

Tray 200 may include a further outer layer or coating 210 made of material selected to prevent tray 200 from adhering to the lips or cheeks a predetermined period of time, such as about ½ an hour.

The predetermined period may be sufficiently long so that a predetermined amount of encapsulated whitener 64 will have been released and will have acted on tooth stains in a manner analogous to the operation of the embodiments of FIGS. 1-5.

This FIG. 6 mouth tray embodiment may be in the form of a strip also.

Examples of Compositions

The following formulation sets forth the percentages of the ranges of materials which may be mixed together (e.g. dissolved in the ethyl alcohol described below) in order to provide the following example of the inventive mixture. After sufficient mixing and entraining of the whitening compound(s) has been achieved, the inventive exemplary mixture and mixture range set forth below for producing the starting material may be dried in a desired final form.

It is contemplated that the starting material or mixture may be dried in a sheet or block form, and then cut or formed into the desired tooth whitening strips, for example, in an additional processing step.

Example of formulation of starting material in which whitening agent is contained, from which starting material the dried or freeze-dried hydrogel in which a whitening agent is entrained or encapsulated is produced.

| (percent by weight) | | |
|---|---|---|
| Range | Example | |
| .001-75% | 25% | Acemannan Hydrogel |
| .001-5% | .5% | Hydroxypropylcellulose - Thickening Agent |
| .001-5% | .5% | Polyvinylpyrrolidone (PVP) - Surfactant |
| .001-75% | 62.5% | Ethyl Alcohol |
| .001-10% | .5% | Simethicone - Antifoaming Agent |
| .001-30% | 11% | Carbamide Peroxide |

Suppliers include, for example:
Acemannan Hydrogel from Aloe Vera L
Supplied by Carrington Labs (2001 Walnut Hill Lane Irving, Tex. 75038)

While this invention has been described as having a preferred design, it is understood that it is capable of further modifications, and uses and/or adaptations of the invention and following in general the principle of the invention and including such departures from the present disclosure as come within the known or customary practice in the art to which the invention pertains, and as may be applied to the central features hereinbefore set forth, and fall within the scope of the invention or limits of the claims appended hereto.

1. A method of whitening a tooth, comprising:
a) providing a dental device, the dental device being configured for being provided on a user's tooth, the dental device including a dried hydrogel incorporating a whitening compound;
b) applying the dental device to a surface of a user's tooth to be whitened;
c) causing the dental device to remain on the user's tooth a sufficient length of time, so that moisture on the user's tooth causes sufficient hydration of the dried hydrogel, and so that the sufficiently hydrated hydrogel releases the whitening compound onto the user's tooth to be whitened, and the whitening compound thus whitens the user's tooth.

2. Method as in claim 1, wherein:
a) the dental device includes a tray configured to engage a front portion and a rear portion of the user's tooth.

3. Method as in claim 1, wherein:
a) the dental device includes a strip configured to be placed on a surface of a user's tooth.

4. Method as in claim 1, wherein:
a) the dried hydrogel includes a freeze-dried hydrogel which changes from a solid to a sticky gel when contacted with moisture on the user's tooth.

5. Method as in claim 4, wherein:
a) the sticky gel is substantially completely biodegradable within the user's mouth in a period of about 6-8 hours.

6. Method as in claim 1, wherein:
a) the dental device includes an inner layer and an outer layer, and the outer layer dissolves in the user's mouth at a different rate than the inner layer.

7. Method as in claim 1, wherein:
a) the whitening agent includes an oxidizing agent.

8. Method as in claim 2, wherein:
a) the whitening agent includes an oxidizing agent encapsulated in a biodegradable substance.

9. Method as in claim 1, wherein:
a) the whitening agent includes an oxidizing agent encapsulated in a biodegradable substance.

10. Method as in claim 3, wherein:
a) the dental device includes a phase transfer catalyst.

11. Method as in claim 1, wherein:
a) the dental device includes a phase transfer catalyst.

12. Method as in claim 3, wherein:
a) a biodegradable coating is provided on an outer surface of the strip and located so that the biodegradable coating prevents sticking of the strip to the user's lips and cheeks.

13. Method as in claim 2, wherein:
a) a biodegradable coating is provided on an outer surface of the tray and located so that the biodegradable coating prevents sticking of the tray to the user's lips and cheeks.

14. Method as in claim 3, wherein:
a) the whitening agent includes hydrogen peroxide incorporated within the dried hydrogel.

15. Method as in claim 2, wherein:
a) the whitening agent includes hydrogen peroxide incorporated within the dried hydrogel.

16. Method as in claim 3, wherein:
a) the dried hydrogel includes acemannan.

17. Method as in claim 2, wherein:
a) the dried hydrogel includes acemannan.

18. Method as in claim 1, wherein:
a) the dried hydrogel includes a material which promotes healing.

19. Method as in claim 18, wherein:
a) the material which promotes healing includes acemannan.

20. Method as in claim 1, wherein:
a) the dried hydrogel is formed from a starting material including the following composition (percent by weight) range:

| | |
|---|---|
| .001-75% | Acemannan Hydrogel |
| .001-5% | Hydroxypropylcellulose - Thickening Agent |
| .001-5% | Polyvinylpyrrolidone (PVP) - Surfactant |
| .001-75% | Ethyl Alcohol |
| .001-10% | Simethicone - Antifoaming Agent |
| .001-30% | Carbamide Peroxide. |

21. Method as in claim 20, wherein:
a) the dried hydrogel is formed from a starting material including the following composition (percent by weight):

| | |
|---|---|
| 25% | Acemannan Hydrogel |
| .5% | Hydroxypropylcellulose - Thickening Agent |
| .5% | Polyvinylpyrrolidone (PVP) - Surfactant |
| 62.5% | Ethyl Alcohol |
| .5% | Simethicone - Antifoaming Agent |
| 11% | Carbamide Peroxide. |
